(19) United States
(12) Patent Application Publication
(10) Pub. No.: US 2026/0334858 A1
(43) Pub. Date: Oct. 29, 2026

(54) AUTOMATIC SPINAL DECOMPRESSION TREATMENT USING DIAGNOSTIC DATA TO OBJECTIVELY DETERMINE PATIENT DIAGNOSTICS AND MEASURE TREATMENT EFFICACY

(71) Applicant: Wizbiz, LLC, Grand Haven, MI (US)

(72) Inventors: Donald W. Wisner, Grand Haven, MI (US); Terry L. Plumert, Grand Haven, MI (US)

(21) Appl. No.: 19/656,883

(22) Filed: Apr. 23, 2026

Related U.S. Application Data
(60) Provisional application No. 63/793,188, filed on Apr. 23, 2025.

(51) Int. Cl. A61B 5/00; A61B 90/00

(52) U.S. Cl. CPC A61B 5/4566; A61B 5/4836; A61B 90/06; A61B 2090/061

(57) ABSTRACT

A system and method for use in adjustment in performing automated distraction/decompression measurement treatments for a cervical or lumbar spine where a prescribed pulling force, speed of force, dwell, and treatment time, and preload forces are entered into a force connect instrument (FCI). The distance associated with each respective pull over the total treatment time is then measured. Patient data is collected and utilized to generate an objective clinical measurement of the overall instant treatment using a baseline for clinical comparison for on-going patient treatments.

### FIELD OF THE INVENTION

The present invention relates to using precise measurement and control systems to apply a prescribed automated spinal decompression treatment while precisely controlling specific treatment parameters and measuring an uncontrolled variable in order to capture objective evidence to determine patent diagnostics and evaluate of treatment efficacy.

### BACKGROUND

Spinal distraction/decompression is generally recognized as an effective treatment for a variety of spinal maladies. Currently, there are a variety of instruments on the market designed for spinal decompression. Most, if not all such equipment positions the patient face up, or in a sitting position, thus voiding the opportunity for any conventional manual manipulation. These instruments are designed so that the patient's upper torso is secured to prevent slippage in the direction of the applied force, and a constant force is applied by pulling on a harness or other contraption designed to attach to a patient's lower extremity in a fashion to transfer the applied force longitudinally to the patient's spine. The intent of these instruments is to apply force to the patient's spine in a fashion to increase the intervertebral distance and produce fluid flow through the incremental spine vertebra, thus reducing the pressure between vertebrae and consequently reducing issues of spinal pain.

Additionally, current instruments that are designed for spinal decompression/distraction use a prescribed force to pull at a prescribed/standard rate until the prescribed force is achieved and then dwell at this force level for a prescribed/standard time before returning to the starting point and repeating the process for a prescribed time. Other than patient feedback, there is no method of objectively determining a patient diagnosis or evaluating the treatment efficacy per treatment as well as over a period of treatments. Thus, new solutions are needed to objectively measure treatment efficacy and relative treatment progress over a series of treatments.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a force connect instrument (FCI) used to apply and measure forces in accordance with an embodiment of the invention.

FIG. 2 is a flow chart diagram illustrating a method for performing automated spinal decompression/distraction measurement treatments for the cervical and lumbar spine.

FIG. 3 is graph illustrating an example of data generated by the FCI and related software to reliably measure progress of the patient.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to as system and method for use in spinal decompression or spinal treatment by performing automated and/or unattended distraction/decompression measurement treatments for the cervical or lumbar spine. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

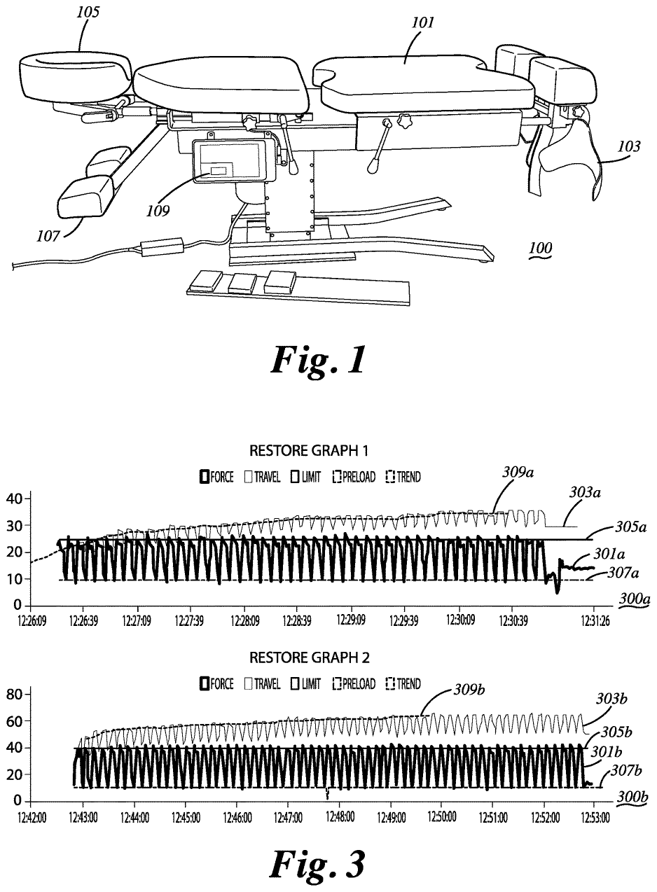

FIG. 1 illustrates a force connect instrument (FCI) used to apply and measure forces in accordance with an embodiment of the invention. The FCI 100 is used to hold, all but one parameter, at an input constant and then measure the variability of the remaining data point. In use the FCI enables the applied force as well as the dwell, speed, and treatment times, that are applied to a patient, to remain at known values for each treatment. Thus, a variable distance of each respective pull is measured with the summation of total distance pulled becomes a defining factor for treatment diagnostics and treatment efficacy. Likewise, the variable could be the applied force while pull distance, dwell, speed, and treatment time are the prescribed constants and the sum or differential force can be a measurement used for patient diagnostics and treatment efficacy. This variable measurement methodology allows monitoring of treatment progress and provides an “end-of” treatment data picture for each patient that coincides with their pain free or acceptable back status. Thus, a reference point for future treatments is created. As seen in FIG. 1, the moveable part of the FCI 100 is the lumbar section 101. Ankle rests/restraints 103 can travel with the full lumbar 101 section during treatment.

Those skilled in the art will recognize that the FCI 100 is similar to most chiropractic tables in that the patient is placed on the FCI face down; with a defined cradle 105 for the face/head. An armrest 107 is used to accommodate arms and hands. Adjustability in the cradle 105 and ankle restraints 103 are used to adapt to various patient heights. Because of this versatility, the patient can receive a multitude of manual chiropractic adjustments using the FCI 100.

The major complement of the FCI 100 is its ability to perform automated distraction/decompression treatments for both the cervical and lumbar spine. Thus, the FCI 100 is designed to provide decompression treatment for most patients whose upper torsos are not restrained. Spinal loads are applied by securing the patient's ankles via straps and applying a longitudinal load pulling force. Securing the upper torso is only necessary in situations where the longitudinal load is high enough to produce some slippage that physically moves/repositions the patient in the direction of the load. In such situations, a thoracic belt (not shown) can be applied to stabilize the patient.

The full treatment consists of entering the prescribed force, speed of force, dwell, and treatment time, and preload force, and then measuring the distance associated with each respective pull, over the total treatment time. This treatment data is then collected and stored as objective evidence used for patient diagnostics and treatment efficacy. In one embodiment, the summation of distances is multiplied by the force to generate a spinal health number (SHN). The SHN can then work as an objective clinical measurement of the overall instant treatment. It can provide a baseline for clinical comparison for on-going treatments to the same patient.

In order for the FCI to make repeated and consistent automated distraction/decompression treatments, a prescription card writer/reader 109 can be used to write, read and/or store FCI usage parameters and data. The FCI parameters are collected and stored on a memory or “prescription” card. The prescription card is a magnetic stripe reader (MSR) card, or the like, that stores collected FCI parameters, settings and other machine data for that patient. No patient clinical data is stored on the prescription card. The prescription card can also include a QR or bar code which is read by the FCI so a patient's treatment can be consistently set-up and repeated by any FCI machine regardless of location. This enables the FCI to read a settings prescription from the prescription card allowing it to introduce and/or use the stored FCI settings. Thus, the FCI can then apply a dependable, reliable and identical automated treatment to the patient regardless of the FCI/patient location.

Those skilled in the art will further recognize that clinicians should be encouraged to maintain the same treatment over several appointments. Since the amount of applied force can be fixed on each patient at each appointment, the clinician can then better determine and/or formulate a diagnosis for each patient and more easily measure treatment efficacy. In one embodiment, a patent may hold and carry their own prescription card that can be read by reader 109. In this way, no matter the location of the FCI, a patient can get new and consistent treatments for continuing their rehabilitation.

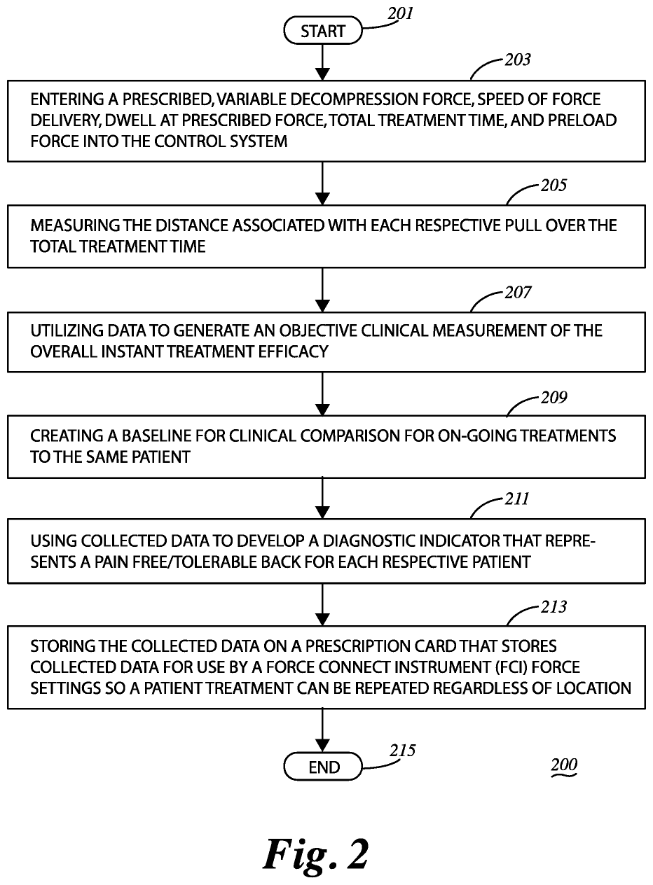

FIG. 2 is a flow chart diagram illustrating a method for performing automated spinal decompression/distraction measurement treatments for the cervical and lumbar spine. The method 200 begins 201 where prescribed parameters are entered 203, such as variable decompression force, speed of force delivery, dwell, total treatment time, and preload force, into the control system. A distance associated with each respective pull is measured 205 over the total treatment time and this data is utilized 207 to generate an objective clinical measurement of the overall instant diagnostic and treatment efficacy. A baseline can then be generated 209 for clinical comparison for on-going treatments to the same patient. This collected data is used to develop 211 a diagnostic indicator that represents a pain free/tolerable back for each respective patient. The collected data is then stored 213 on a prescription card used to store collected prescription data such as force settings and the like for use by a force connect instrument (FCI) so a patient's treatment can be repeated regardless of location. Thereafter the process ends 215.

FIG. 3 is graph illustrating an example of data generated by the FCI and related software to reliably measure progress of the patient. The graphs 300a, 300b show various representations indicating use of the FCI such as force 301a, 301b, travel 303a, 303b, limit 305a, 305b, preload 307a, 307b and trends of a treatment 309a, 309b. These parameters and diagnostics can be easily measured to determine patient diagnosis, treatment repeatability, progress, results, and/or overall efficacy.

Thus, embodiments of the present invention are directed to a system and method for performing automated spinal decompression/distraction measurement treatments for the cervical and lumbar spine. The system and method comprise the steps of entering a prescribed, variable decompression force, speed of force delivery, dwell at prescribed force, total treatment time, and preload force into the control system for a force connect instrument (FCI). The distance associated with each respective pull measured over the total treatment time and the data is used to generate an objective clinical measurement of the overall instant treatment efficacy. A baseline is created for clinical comparison for on-going treatments to the same patient where the collected data is used to develop a diagnostic indicator that represents a pain free/tolerable back for each respective patient. The collected data is written to a prescription card that stores collected data for use by the FCI so a patient treatment can be repeated regardless of location.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A method for performing automated spinal decompression/distraction measurement treatments for the spine comprising the steps of:
entering a prescribed decompression force, speed of force delivery, dwell at prescribed force, total treatment time, and preload force into the control system;
measuring the distance associated with each respective pull over the total treatment time;
utilizing data to generate an objective clinical measurement of the overall instant treatment efficacy; and
creating a baseline for clinical comparison for on-going treatments to the same patient.

2. A method as in claim 1, where the treatments are used for both the cervical or lumbar spine.

3. A method as in claim 1, where decompression force is used as a variable.

4. A method as in claim 1, further comprising the step of:
storing the collected data on a prescription card for use by a force connect instrument (FCI) for future treatments.

5. A method as in claim 4, where the prescription card enables a repeatable patient treatment using different FCI machines regardless of location.

6. A method as in claim 1, further comprising the step of:
using collected data to develop a Spine Health Number (SHN) that represents a pain free/tolerable back for each respective patient.

7. A method as in claim 3, where the SHN is used as a benchmark for future treatments.

8. A method for performing automated spinal decompression/distraction measurement treatments for the cervical and lumbar spine, comprising the steps of:
entering a prescribed, variable decompression force, speed of force delivery, dwell at prescribed force, total treatment time, and preload force into the control system;
measuring the distance associated with each respective pull over the total treatment time;
utilizing data to generate an objective clinical measurement of the overall instant treatment efficacy;
creating a baseline for clinical comparison for on-going treatments to the same patient; and
using collected data to develop a diagnostic indicator that represents a pain free/tolerable back for each respective patient.

9. A method as in claim 7, further comprising the step of:
storing the collected data on a prescription card that stores collected data for use by a force connect instrument (FCI).

10. A method as in claim 9, where the prescription card enables a repeatable patient treatment using different FCI machines regardless of location.

11. A method as in claim 6, wherein the diagnostic indicator represents a Spine Health Number (SHN).

12. A method as in claim 8, further comprising the step of:
storing collected FCI data on a portable memory device to act as a prescription for repeated treatments regardless of patient or FCI location.

13. A method for performing automated spinal decompression/distraction measurement treatments for the cervical and lumbar spine, comprising the steps of:
entering a prescribed, variable decompression force, speed of force delivery, dwell at prescribed force, total treatment time, and preload force into the control system;
measuring the distance associated with each respective pull over the total treatment time;
utilizing data to generate an objective clinical measurement of the overall instant treatment efficacy;
creating a baseline for clinical comparison for on-going treatments to the same patient;
using collected data to develop a diagnostic indicator that represents a pain free/tolerable back for each respective patient; and
storing the collected data on a prescription card that stores collected data for use by a force connect instrument (FCI) so a patient treatment can be repeated regardless of location.

14. A method for performing automated spinal decompression/distraction measurement treatments as in claim 13, wherein the collected data includes FCI force settings for an individual patient.
